(19) United States
(12) Patent Application Publication
(10) Pub. No.: US 2023/0416131 A1
(43) Pub. Date: Dec. 28, 2023

(54) Water Purification System With Inline Measurement Of Total Organic Carbon And Method Of Operating Such System

(71) Applicant: Merck Patent GmbH, Darmstadt (DE)

(72) Inventors: Xavier Percevault, Molsheim (FR); Didier Meyer, Molsheim (FR)

(21) Appl. No.: 18/038,265

(22) PCT Filed: Nov. 23, 2021
(86) PCT No.: PCT/EP2021/082570

§ 371 Date: May 23, 2023

(30) Foreign Application Priority Data
Nov. 26, 2020 (EP) 20306447.2

(51) Int. Cl. C02F 9/00

(52) U.S. Cl. CPC C02F 9/00; C02F 1/325

(57) ABSTRACT

The present application relates to a water purification system and method with an inline measurement of total organic carbon (TOC) content.

### TECHNICAL FIELD

The present application relates to a water purification system and method with an inline measurement of total organic carbon (TOC) content.

### BACKGROUND

In applications such as in the pharmaceutical, life science or semiconductor areas, natural water or water coming from the faucet (“tap water”) may not be sufficiently pure as it may contain contaminants that may cause, for example, undesired side reactions and/or negatively influence reproducibility in analytical or production processes. Thus, depending upon the targeted application, water purity has to be improved by removing the contaminants comprised in the water either partly or even as much as possible. The highest purity water, often denoted as “ultrapure” or in accordance with ASTM D 1193-06 as “Type I” water, is, for example, characterized by a resistivity of at least 18.0 MΩ·cm and at most 5 ppb of total organic carbon (TOC).

Water purification systems allowing to produce ultrapure or Type I water are known as such. An integral water purification system designed to purify water from tap water comprises various water purification steps such as, for example, filtration, reverse osmosis, electrodeionization, UV radiation treatment, ion exchange steps. Generally, such purification system comprises a first purification stage, wherein tap water is purified to a first purity grade (for example, Type 2 or lower as defined by ASTM D 1193-06), and a second purification stage, wherein the pre-purified water from the first purification stage is further purified to a higher purity (for example, Type 1 as defined by ASTM D 1193-06), which can then be dispensed from the system and used.

Purifying water to the ultrapure level is a challenge due to the acceptable levels of contaminants being very low. Thus, to avoid build-up of contaminants in the water purification system and the dispensing section, for example, when no water is dispensed from the system, the purified water needs to be continuously re-circulated throughout the system, for example, by continuous re-circulation through the first and second purification stages.

To be sure that the dispensed water corresponds to the desired purity, users have an interest in a purification system that is able to continuously or at least regularly monitor a number of properties of the purified water. While some of these properties are relatively easy to measure, it has proven to be rather challenging to reliably determine total organic carbon (TOC) content.

U.S. Pat. No. 5,677,190 A discloses a measurement cell and circuit for measuring the electrical characteristics of a liquid sample during exposure to UV radiation. In one implementation for the measurement of TOC in water by oxidation to CO₂, the UV radiation is generated by a low pressure mercury lamp. The water sample in the measurement cell is irradiated until conductivity no longer increases, meaning that all organic components originally present in the water have been oxidized. The difference in conductivity before and after irradiation is then computed into total organic carbon content. While this method is very precise, it is rather time-consuming and also requires additional equipment specifically dedicated to the determination of TOC content.

U.S. Pat. No. 5,272,091 A discloses a method for predicting the organic carbon content of water discharged from a water purification system, wherein water is directed from a water source past, for example, an ultraviolet light source comprised in the water purification system. Then, using a reference-mode resistivity change in combination with a purification-mode resistivity the value for the purified water is obtained, which in association with the carbon content of water discharged from the water purification system allows predicting the carbon content of water discharged from the water purification system.

EP 0 498 888 A1 discloses a method for measuring the amount of total organic matters in an ultrapure water, the method applying ultraviolet rays to ultrapure water whose specific resistance value has been set to a known constant one by ion exchange treatment, finding decreases by subtracting specific resistance values of ultrapure water measured continuously at different points from the abovesaid constant specific resistance value, and giving total organic carbon content on the basis of the correlation of the decreases with the quantity of generated CO₂.

Though the methods disclosed in U.S. Pat. No. 5,272,091 A and EP 0 498 888 A1 offer an economic and simple way to use the equipment already present in the water purification system to determine the TOC content, there is still room for improvement because, with the TOC content being determined directly after (i.e. downstream of) the UV radiation source but before (i.e. upstream of) the second purification step, the value thus obtained may not always correspond to the actual TOC content of the water dispensed from the water purification system. Furthermore, these methods can lead to false alarms for higher than desired levels of TOC when the first purification stage is no longer fully performant but such lack of performance is compensated for by the second purification stage.

Thus, there is a need in industry for an improved purification system and method allowing to determine the total organic carbon (TOC) content at the point of dispensing the ultrapure water from the water purification system, preferably such purification system and method not requiring the installation of additional, possibly technically complicated and/or expensive, components in the water purification system. Preferably, such purification system and method should not lead to an increase in maintenance requirements either.

### SUMMARY

The present inventors have now surprisingly found that the above objects may be attained either individually or in any combination by the water purification system and method of the present application.

The present application therefore provides for a water purification system (100) comprising
(a) a purification stage (104) comprising, preferably essentially consisting of, in sequence a UV radiation treatment step (105) and a polisher (107); and
(b) a device (106) for measuring total organic carbon content, the device located downstream of the UV radiation treatment step (105) and upstream of the polisher (107),
(c) a measurement loop,
   wherein the measurement loop comprises the water purification stage (104) and the device (106) for measuring total organic carbon.

The present application therefore also provides for a process for operating a water purification system (100), the water purification system (100) comprising
(a) a purification stage (104) comprising, preferably essentially consisting of, in sequence a UV radiation treatment step (105) and a polisher (107); and
(b) a device (106) for determining total organic carbon content, the device located downstream of the UV radiation treatment step (105) and upstream of the polisher (107),
(c) a measurement loop,
wherein the measurement loop comprises the water purification stage (104) and the device (106) for measuring total organic carbon,
wherein the process comprises the steps of
(1) passing water through the purification stage (104), thereby obtaining purified water; and
(2) passing the purified water through the measurement loop and determining by means of the device (106) the content of total organic carbon comprised in the purified water

### BRIEF DESCRIPTION OF THE DRAWINGS

FIG. 1 shows an exemplary schematic representation of the present water purification system comprising a connecting line C1 and a two-way or three-way valve (111).

FIG. 2 shows an exemplary schematic representation of the present water purification system comprising a connecting line C1 and two valves (111a) and (111b) in conjunction.

FIG. 3 shows an exemplary schematic representation of the present water purification system comprising a connecting line C2.

FIG. 4 shows a schematic exemplary representation of a preferred water dispensing section (109) together with device (108) for determining total organic carbon content, valve (110), and part of the recycle line (R).

FIG. 5a shows an exemplary schematic representation of the flow of water through the present water purification system comprising a connecting line C1 and a two-way or three-way valve (111) in dispensing mode.

FIG. 5b shows an exemplary schematic representation of the flow of water through the present water purification system comprising a connecting line C1 and a two-way or three-way valve (111) in measuring mode.

FIG. 5c shows an exemplary schematic representation of the flow of water through the present water purification system comprising a connecting line C1 and a two-way or three-way valve (111) in re-cycling mode.

FIG. 6a shows an exemplary schematic representation of the flow of water through the present water purification system comprising a connecting line C2 in dispensing mode.

FIG. 6b shows an exemplary schematic representation of the flow of water through the present water purification system comprising a connecting line C2 in measuring mode.

FIG. 6c shows an exemplary schematic representation of the flow of water through the present water purification system comprising a connecting line C2 in re-cycling mode.

FIG. 7 shows the total organic carbon content (TOC) for the water coming from the first purification stage (see continuous line) and for dispensed water (see dashed line) for Example 1.

FIG. 8 shows resistivity curves and TOC curves for Example 2.

FIG. 9 shows resistivity curves and TOC curves for Example 4.

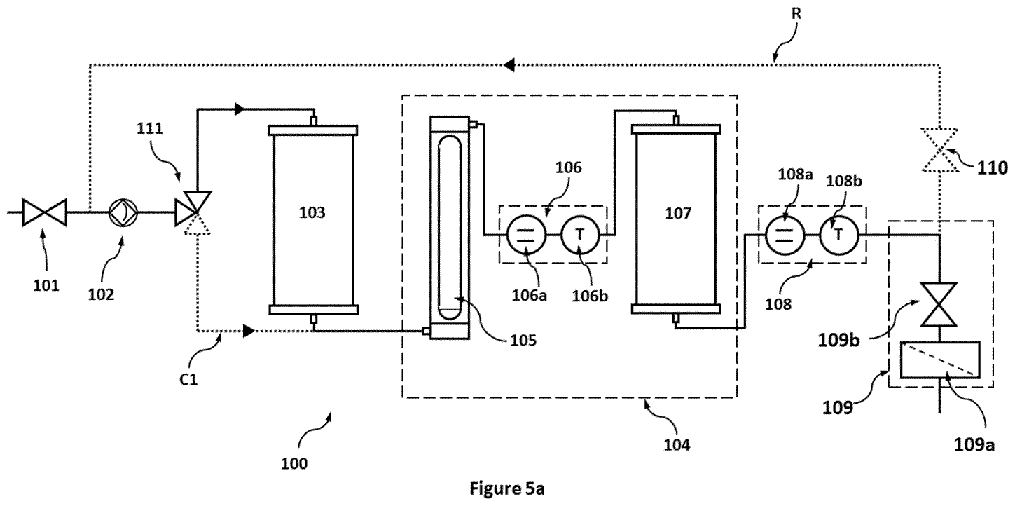

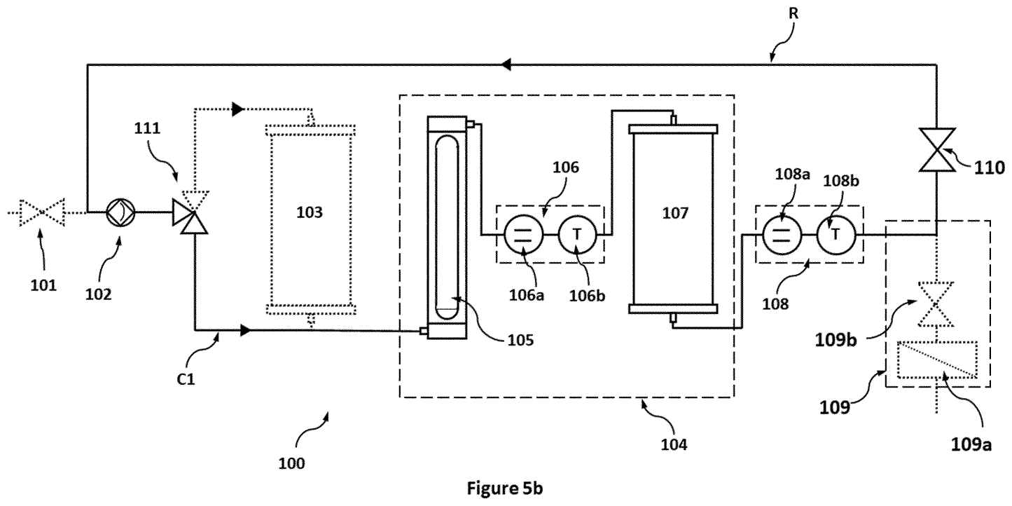

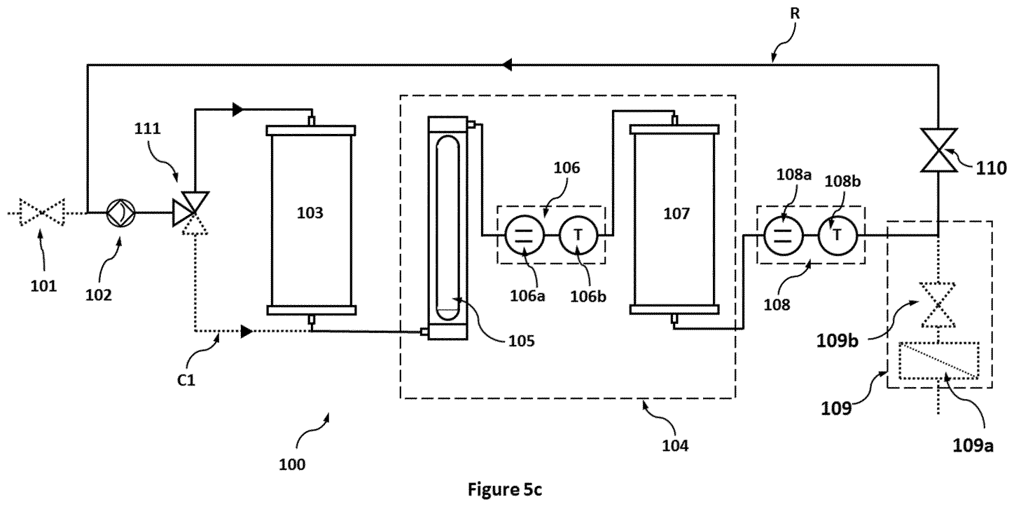

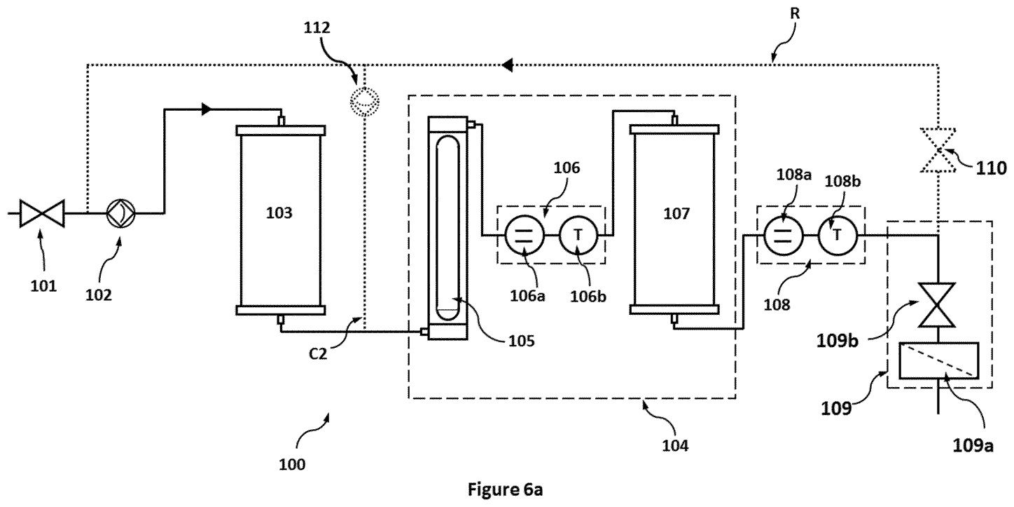

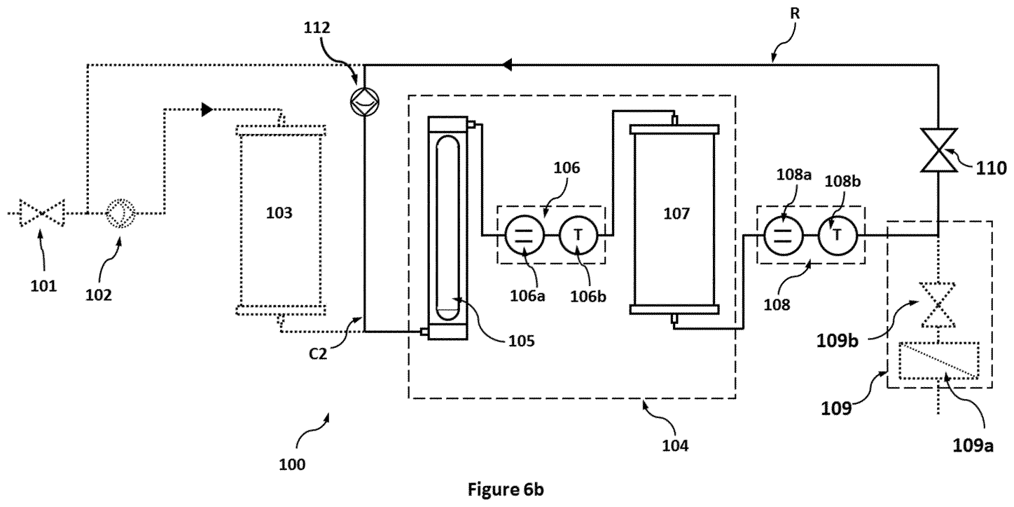

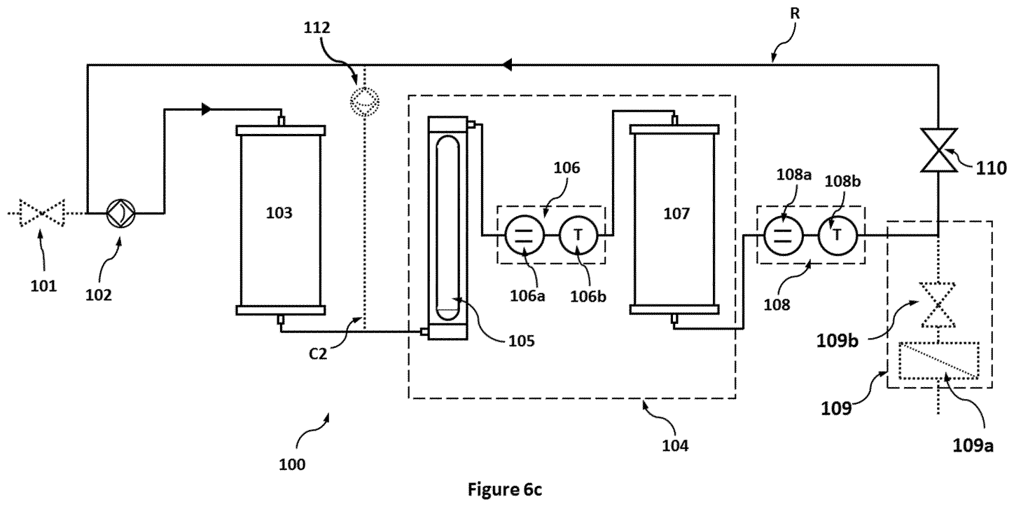

In FIGS. 5a, 5b, 5c, 6a, 6b, and 6c dotted lines and symbols indicate components of the respective depicted water purification systems that are not in use in the particular mode represented in the respective figure.

Throughout the present application, including the figures, identical components etc. are indicated by corresponding reference numerals.

In the figures, arrows are used to indicate the general direction of the flow of water through the present water purification system.

### DETAILED DESCRIPTION

As used herein, the term “upstream” is used to indicate a direction opposite the flow of water in the water purification system, i.e. indicates a direction towards the water inlet. Expressed differently, going upstream indicates a direction from the water dispensing section to the water inlet.

As used herein, the term “downstream” is used to indicate a direction with the general flow of water in the purification system, i.e. indicates a direction towards the water outlet. Expressed differently, going downstream indicates a direction from the water inlet to the water dispensing section.

As used herein, in the context of the water purification stages, the terms “essentially consist of”, “essentially consists of”, and “essentially consisting of” are used to denote that the respective stage does not comprise further means for water purification (such as means for removing contaminants comprised in the water), but may nevertheless comprise one or more auxiliary devices, which may, for example, be selected from the non-limiting group consisting of temperature sensors, flow sensors, pressure sensors, conductivity measuring devices, flow controls, and valves.

As used herein, the terms “water purification flow”, “measurement loop”, and “recycle flow” are used to denote a continuous liquidly connected flow path for the water passing through the present water purification system.

As used herein, the terms “UV” and “ultraviolet” are generally, and if not otherwise limited, used to denote electromagnetic radiation having a wavelength of at least 100 nm and of at most 400 nm.

In general terms, the present application relates to a water purification system for producing ultrapure, i.e. Type I, water. Particularly, the present application relates to a water purification system comprising (a) a purification stage, (b) a device for measuring total organic carbon (TOC) content, and (c) a measurement loop.

The purification stage denoted under (a), which is generally throughout this application for reasons of clarity referred to as “second purification stage”, comprises, preferably essentially consists of, in sequence a UV radiation treatment step and a polisher.

The device for measuring total organic carbon content is located within the second purification stage at a position downstream of the UV radiation treatment step and upstream of the polisher. Such device for measuring total organic carbon content preferably is a device for measuring conductivity, which in short may also be referred to as a “conductivity cell” throughout this application. Such conductivity cell is generally associated with a temperature sensor.

The measurement loop comprises, preferably essentially consists of, in sequence, the UV radiation treatment step and the polisher (i.e. the second purification stage), and the device for measuring total organic carbon content located in between the UV radiation treatment step and the polisher.

A preferred water purification system comprises, preferably essentially consists of, in sequence a water inlet, a water purification flow, and a recycle line (R).

The water inlet, for example comprising a solenoid valve, serves to introduce water from an external source into the water purification system. Water from such external source generally is of lower purity than Type I water and may, for example, come from a public water system (“tap water”) or from a system supplying, for example, deionized water or Type II water. It is noted that for the purposes of the present application the water inlet is considered to represent the most upstream point in the present water purification system.

The water purification flow comprises, preferably essentially consists of, in sequence a pump, a first (or preceding) purification stage, the second purification stage, and a dispensing section, all of which are as defined herein.

The first purification stage purifies water to a first water purity grade, preferably to a first water purity grade that is higher than the water purity grade of the water introduced into the present water purification system via the water inlet described above (“feed water”).

The purification media comprised in the first purification stage is not particularly limited. It may, for example, be selected depending upon the purity of the feed water fed into the present water purification system. Preferably, the first purification stage comprises one or more purification media selected from the group consisting of cationic ion exchanger, anionic ion exchanger, and activated carbon. Thus, the first purification stage preferably reduces the level of ionic contaminants and/or organic contaminants.

Water exiting the first purification stage—if the first purification stage is fully functional—is supposed to have a resistivity of up to 18.2 MΩ·cm at 25° C. and a very low level of organic contaminants.

Preferably, the second purification purifies the water to a second water purity grade higher than the first water purity grade.

Preferably, the second purification stage comprises, preferably essentially consists of, in sequence the UV radiation treatment step and the polisher.

First and second purification stages, and for the second purification stage particularly the polisher, are preferably disposable devices, e.g. disposable cartridges, comprising the respective purification media as defined above.

Preferably, the UV radiation treatment device comprises a UV radiation source emitting radiation, preferably having a wavelength of at least 150 nm, such UV radiation source preferably being selected from the group consisting of low-pressure mercury lamp, cold cathode mercury lamp, and excimer lamp.

In such UV radiation treatment device, the UV radiation will oxidize organic contaminants at least partly, leading to the generation of ions, particularly carbonate ions (CO₃²⁻) or hydrogen carbonate ions (HCO₃⁻). The presence and particularly the concentration of such ions may then be determined using a conductivity cell with associated temperature compensation, with the temperature of the water being detected by a respective temperature sensor. Taking the increase in conductivity, which is due to the additional ions generated by the oxidation of the organic contaminants in the water, the concentration/level of organic contaminants in the water can then be calculated using a dedicated algorithm. This algorithm is not particularly limited. Further details on the determination of total organic carbon content may be found in WO 2018/153822 A1, incorporated herein by reference. It is, however, noted that such determination and calculation is well known in the art and generally need not be explained in detail.

It is noted that besides using UV radiation the organic contaminants comprised in the water may be oxidized by any method selected from the non-limiting list consisting of thermal oxidation, chemical oxidation (e.g. by adding a peroxide, such as hydrogen peroxide), UV-persulfate oxidation, and any combination of these. It is noted that though the present description uses UV radiation when illustrating the present water purification system and method, such description also applies to the other methods as well. Nevertheless, for the present water purification system and method, oxidation by UV radiation is preferred.

Preferably, the polisher comprises any one or more purification medium selected from the group consisting of activated carbon, cationic ion exchanger, and anionic ion exchanger.

Preferably, downstream of the polisher and upstream of the dispensing section, the purified water again passes through a conductivity cell with associated temperature sensor to determine its conductivity, consequentially its total ion concentration, and thus its purity and suitability for the intended purpose. Thus, this conductivity cell—if present—also allows the user to assess the good overall functioning of the present water purification system.

The dispensing section allows withdrawal of the ultrapure water produced by the present water purification system through one or more water outlet. The one or more water outlet may be directly located at or may be located further removed, for example, a few meters, from the remainder of the water purification system. If more than one outlet is present, they may be arranged in sequence or in parallel, but are preferably arranged in parallel. Preferably, each water outlet comprises a dispensing valve allowing to control the dispensing rate and volume of the purified water. The dispensing valve(s) may be a solenoid valve of the type with a normally closed flow path. Further, each outlet preferably comprises a “by-pass” allowing water to be circulated through the dispensing section even when no purified water is dispensed from said water outlet, for example when the present water purification system is in recycling mode. Such “by-pass” may thus be considered part of recycle line (R), or may be considered as part of the combined water purification flow and recycle flow/recycle line (R), for example when the present water purification system is in recycling mode. Exemplary water outlets are, for example, disclosed in EP 1 814 007 A1.

Preferably, at the point of use, i.e. upstream of a purified water outlet comprised in the dispensing section, the dispensing section comprises a filter element, which preferably comprises a 0.22 μm membrane or any other suitable purification means. Such filter element is preferably adapted to eliminate trace contaminants, such as bacteria, pyrogens, endocrine disruptors, and/or volatile organic compounds to only give some non-limiting examples.

The recycle line (R) liquidly connects the water purification flow from a location downstream of the dispensing section to a location downstream of the water inlet and upstream of the pump.

The water purification flow and/or the recycle line (R) are adapted to allow by-passing the first purification stage, thereby establishing the measurement loop.

Such by-pass may be achieved by the present water purification system comprising a connecting line C1 liquidly connecting the water purification flow from a branching point, which is located downstream of the pump and upstream of the first purification stage, to a location downstream of the first purification stage and upstream of the second purification stage, particularly upstream of the UV radiation treatment stage.

Thus, the present water purification system preferably comprises a two-way valve or a three-way valve located in the water purification flow at the branching point. This valve allows to control the flow of water along the water purification flow or along connecting line C1 thereby allowing to by-pass the first purification stage.

Alternatively, the present water purification system preferably comprises two valves in conjunction, with the first valve being located in the water purification flow downstream of the branching point and upstream of the first purification stage, and the second valve being located in connecting line C1. Thus, by closing the first valve and opening the second valve the flow of water goes directly from the pump through the connecting line C1 to the UV radiation treatment step of the second purification stage. Inversely, by opening the first valve and closing the second valve the flow of water goes to and through the first purification stage and then on to the second purification stage. Said first valve may, for example, be a 2-way valve. Said second valve may, for example, be a 2-way valve or a check valve.

Optionally, in addition to the two valves used in conjunction, a check valve may be placed in the water purification flow at a location downstream of the pump and upstream of the branching point. The check valve blocks the back-flow of water, i.e. inhibits water from flowing in the direction of the pump and the water inlet.

Alternatively, such by-pass may be achieved by the present water purification system comprising a connecting line C2 liquidly connecting the recycle line (R) to the water purification flow at a location downstream of the first purification stage and upstream of the second purification stage, particularly upstream of the UV radiation treatment stage. Preferably connecting line C2 comprises a circulation pump.

The present application also relates to a process for operating a water purification system, the water purification system comprising (a) a purification stage, which is generally throughout this application for reasons of clarity referred to as “second purification stage”, (b) a device for determining total organic carbon content, the device as described herein located downstream of the UV radiation treatment step and upstream of the polisher, and (c) a measurement loop as defined herein, wherein the process comprises the steps of (1) passing water through the purification stage, thereby obtaining purified water; and (2) passing the purified water through the measurement loop and determining by means of the device the content of total organic carbon comprised in the purified water.

Preferably the present application relates to a process for operating a water purification system, the water purification system comprising (a′) a water inlet; (b′) a water purification flow as defined herein; and (c′) a recycle flow (R) as defined herein, the process comprising the steps of
(1′) providing water via the water inlet;
(2′) passing water through the water purification flow, thereby obtaining purified water;
(3′) dispensing the purified water obtained in step (2′); and
(4′) determining the level of total organic compound by passing purified water through the measurement loop by by-passing the first purification stage and determining total organic carbon content by means of the device for determining total organic carbon content.

Following step (3′) of dispensing the purified water obtained in the preceding step (2′), the present water purification system is changed (e.g. manually by an operator), or preferably automatically switches, from dispensing mode to the post-dispensing or measuring mode of step (4′), for example, by closing the dispensing valve and opening the valve in the recycle line (R).

Preferably, in step (4′) of the present process the first purification stage is by-passed by blocking the water purification flow downstream of the branching point and upstream of the first purification stage, and allowing water to directly pass through connecting line C1 to the second purification stage, with the water purification system preferably comprising at the branching point a two-way valve or three-way valve, or a first valve being located in the water purification flow downstream of the branching point and upstream of the first purification stage, and a second valve being located in connecting line C1.

Alternatively, in step (4′) of the present process the first purification stage is preferably by-passed by forcing water through connecting line C2, with connecting line C2 as defined herein.

Preferably, the present process subsequently to step (4′) further comprises re-cycling the purified water by passing it sequentially through the water purification flow and the recycle line (R). The term “re-cycling” is used herein to denote that the water is repeatedly passed through the water purification flow and the recycle line (R). Such re-cycling allows keeping the levels of potential contaminants of the purified water as low as possible, particularly when no purified water is being dispensed.

The present system and method allow determining the total organic carbon content of the purified water at the point of use, i.e. of the water as dispensed, while being able to make use of equipment/devices/components already comprised in the present water purification system and part of the water purification flow. This, in consequence, also allows for a simplified design of the present water purification system as compared to a water purification system of the prior art, wherein total carbon content is determined in the water purification flow downstream of the UV radiation treatment device but upstream of the polisher.

In the following, the present water purification system and the respective process for operating such water purification system are explained in further detail referring to the exemplary schematic drawings. It is noted that the definitions and descriptions given above for the respective components of the present water purification system also apply to the corresponding elements depicted in the figures.

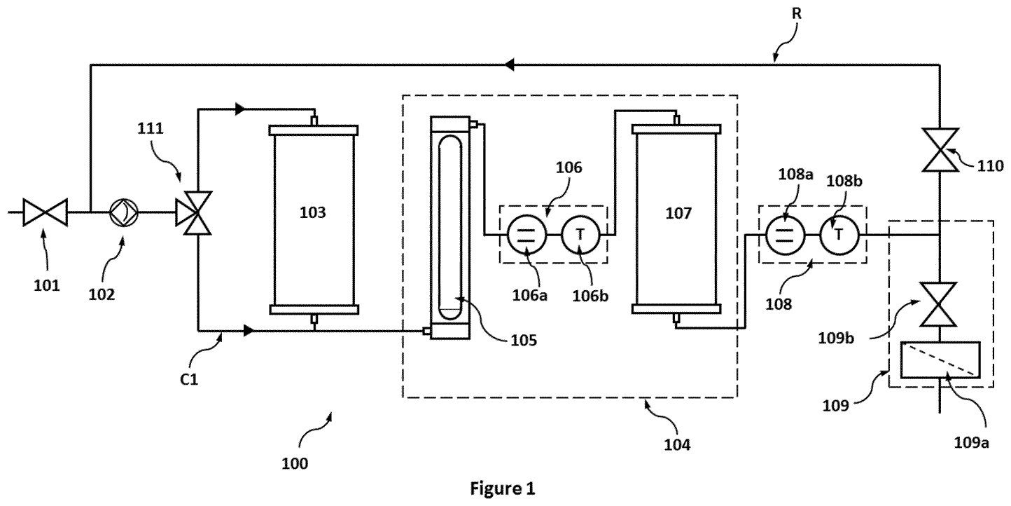

A schematic exemplary representation of a preferred water purification system (100) of the present application is shown in FIG. 1. System (100) comprises, preferably essentially consists of, in sequence water inlet (101), pump (102), first purification stage (103), second purification stage (105), optional device (108) for measuring conductivity (“conductivity cell”), and dispensing section (109). Further, system (100) comprises the recycle line (R).

To be purified water is fed to the water purification system (100) via water inlet (101). It then passes into the water purification flow, which comprises pump (102), the first purification stage (103), the second purification stage (104), yielding the purified ultrapure water, and dispensing section (109), from which the purified water may then be dispensed. Optionally, downstream of the second purification stage (104) and upstream of dispensing section (109), the water purification flow may comprise a conductivity cell (108).

Second purification stage (104) comprises, preferably essentially consists of, in sequence UV treatment stage (105), and polisher (107). In between UV treatment stage (105) and polisher (107), i.e. downstream of UV treatment stage (105) and upstream of polisher (107), the present water purification system (100) comprises a device (106) for determining total organic carbon content.

Device (106) comprises a conductivity measurement cell (106a) and a temperature sensor (106b).

Conductivity cell (108)—if present—located downstream of the polisher and upstream of the dispensing section comprises a conductivity measurement cell (108a) and a temperature sensor (108b),

Dispensing section (109) comprises a water dispensing valve (109b), allowing to open and close the flow of purified water to be dispensed. Preferably, the dispensing section (109) optionally discloses a filtering element (109a) as defined above.

Water purification system (100) also comprises a recycle line (R), through which water can be cycled from dispensing section (109) back upstream to a location between water inlet (101) and pump (102), i.e. downstream of water inlet (101) and upstream of pump (102). Re-cycling purified water through the recycle line (R) and the water purification flow serves to maintain high purity of the already purified water in water purification system (100) when no purified water is dispensed.

Dispensing valve (109b) and valve (110) in combination allow control of the flow of purified water. Opening dispensing valve (109b) comprised in dispensing section (109), while keeping valve (110) in the recycle line (R) closed, allows to dispense purified water from the dispensing section (109). On the other hand, closing dispensing valve (109b) and opening valve (110) allows water to pass into the recycle line (R). The water is then passed through the recycle line (R) back to a location between water inlet (101) and pump (102), i.e. downstream of water inlet (101) and upstream of pump (102). From there it can then pass through the water purification flow.

Further, the present water purification system (100) as shown in FIG. 1 comprises a two- or three-way valve (111) and a connecting line C1 liquidly connecting the two- or three-way valve (111) to the water purification flow at a location downstream of first purification stage (103) and upstream of second purification stage (104). Two- or three-way valve (111) allows to direct the flow of water either along the water purification flow, i.e. to the first purification stage (103), or through connecting line C1, thereby by-passing the first purification stage (103).

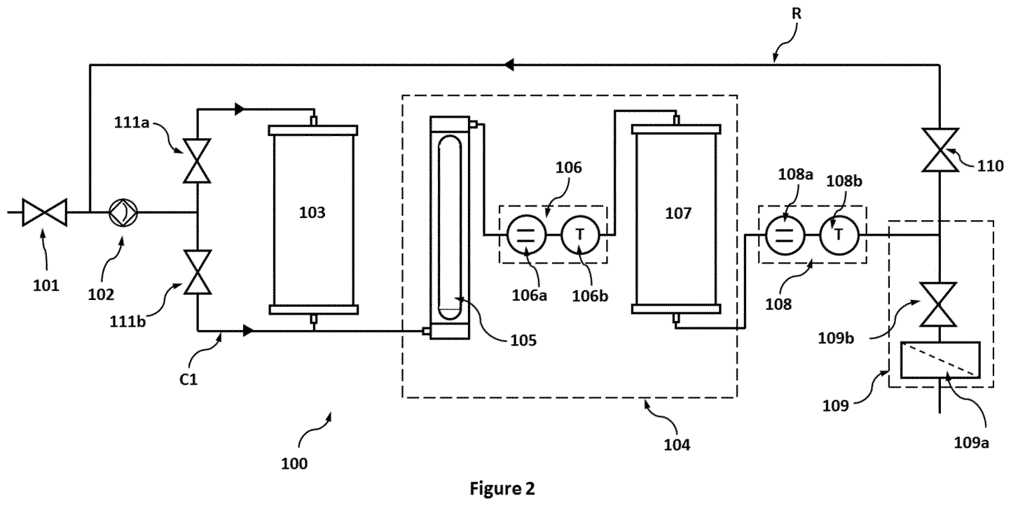

FIG. 2 shows a schematic exemplary representation of another preferred water purification system (100) as defined herein, which differs from the one of FIG. 1 in that two-or three-way valve (111) of the water purification system (100) of FIG. 1 has been replaced by a system of two valves (111a, 111b) working in conjunction to direct the flow of water either along the water purification flow, i.e. to and through the first purification stage (103), or along connecting line C1, thereby by-passing the first purification stage (103). Thus, opening valve (111a) and closing valve (111b) directs the flow of water towards the first purification stage (103). On the other hand, closing valve (111a) and opening valve (111b) directs the flow of the water along connecting line C1 directly to the second purification stage (104), thereby by-passing the first purification stage (103). Valve (111a) may, for example, be a 2-way valve. Valve (111b) may, for example, be a 2-way valve or a check valve.

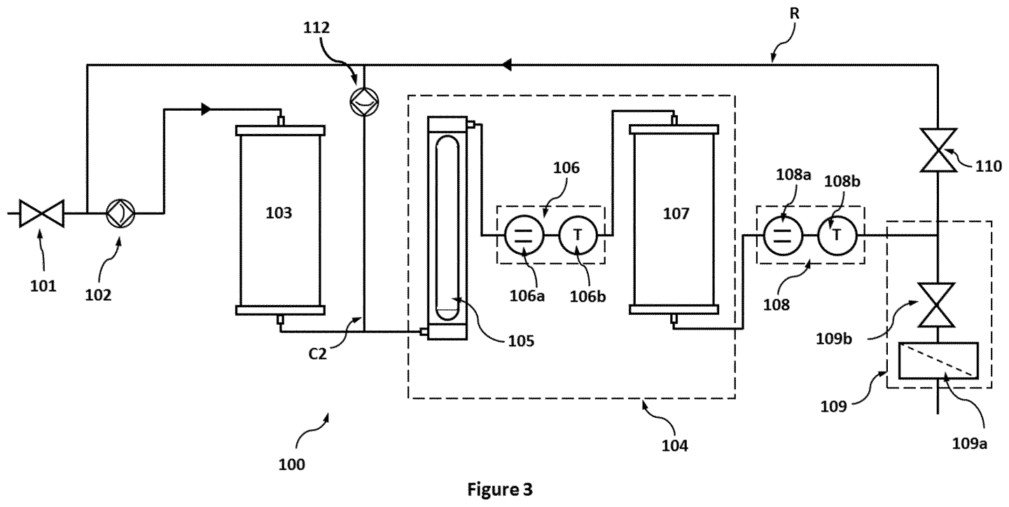

FIG. 3 shows a schematic exemplary representation of another water purification system (100) as defined herein, which in comparison to the water purification system (100) of FIG. 1 comprises a connecting line C2, which in turn comprises a circulation pump (112), instead of two- or three-way valve (111) and connecting line C1.

Optionally the water purification system (100) depicted in FIG. 3 may also comprise a valve in between the branching point of connecting line C2 and the recycle line (R) and the point where the recycle line (R) feeds back into the water purification flow downstream of water inlet (101) and upstream of pump (102).

Preferably, connecting line C2 is connected to the recycle line (R) at a point between the valve (110) and at a location between water inlet (101) and pump (102), or alternatively—if the water purification system (100) depicted in FIG. 3 comprises a valve in between the branching point of connecting line C2 and the recycle line (R) and the point where the recycle line (R) feeds back into the water purification flow downstream of water inlet (101) and upstream of pump (102)—connecting line C2 is connected to the recycle line (R) at a point between the valve (110) and such optional valve.

Turning on circulation pump (112) and turning off pump (102), as done in measuring mode, will deviate water flow from the recycle line (R) into connecting line C2, which then feeds into the water purification flow at a location downstream of the first purification stage (103) and upstream of the second purification stage (104), thereby by-passing the first purification stage.

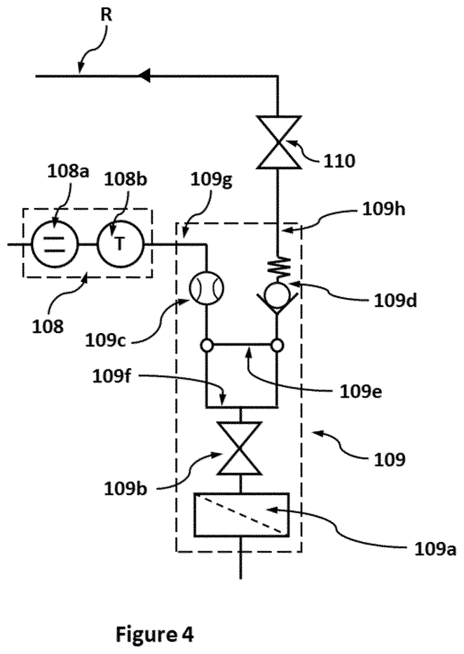

FIG. 4 shows a schematic exemplary representation of a preferred water dispensing section (109) together with optional conductivity cell (108), valve (110), and part of the recycle line (R). Such preferred dispensing system (109) preferably comprises a dispensing flow and a recycle flow. The dispensing flow preferably comprises, more preferably consists of, in sequence a flow meter (109c), which is adapted to allow measuring the volume of dispensed water, line (109f), a dispensing valve (109b), and a filtration element (109a) allowing to remove contaminants from the water to be dispensed as discussed above. The recycle flow is preferably connected to the dispensing flow via connecting line (109e) and/or line (109f). The recycle flow preferably comprises, more preferably consists of, in sequence a check valve (109d) and outlet (109h) connected to the recycle line (R) upstream of valve (110). Furthermore, the dispensing flow of the dispensing section (109) is connected from a location downstream of the flow meter (109c) and upstream of line (109f) to the recycle flow of the dispensing section (109) at a location downstream of line (109f) and upstream of the check valve (109d).

When the present water purification system is in dispensing mode, subsequently to exiting polisher (107) and passing through optional conductivity cell (108), the purified water enters the dispensing section (109) through the dispensing section inlet (109g), passes through the flow meter (109c) and line (109f), and is dispensed via dispensing valve (109b) and filtration element (109a).

When the present water purification system is in measuring and recycling mode, subsequently to exiting polisher (107) and passing through optional conductivity cell (108), the purified water enters the dispensing section (109) through the dispensing section inlet (109g), and is recycled via connecting line (109e) and/or line (109f), the check valve (109d), and the outlet (109h) to the recycle line (R). Thus, the recycle flow may comprise in sequence the following parts of dispensing section (109): dispensing section inlet (109g), and is recycled via connecting line (109e) and/or line (109f), the check valve (109d), and the outlet (109h).

FIGS. 5a, 5b and 5c show a schematic exemplary representation of the water flow in the water purification system (100) of FIG. 1 in dispensing, measuring, and re-cycling mode, respectively. It is noted that in FIGS. 5a, 5b and 5c dispensing section (109), particularly the parts of dispensing section (109) forming part of the recycle flow as described above, are not shown in detail.

FIG. 5a shows a schematic exemplary representation of the water flow of the water purification system (100) of FIG. 1 in dispensing mode. Feed water is fed into water purification system (100) via water inlet (101), from where the water sequentially passes through the first purification stage (103), UV radiation treatment stage (105), device (106) for determining the total organic carbon content, polisher (107), an optional conductivity cell (108), and then into dispensing section (109), from where the purified water is dispensed.

Once dispensing is terminated, water purification system (100) of FIG. 1 is switched (manually or automatically) into measuring mode, which is shown in FIG. 5b, by opening valve (110) and closing dispensing valve (109b), wherein water flows through pump (102), two- or three-way valve (111), connecting line C1 (thereby by-passing the first purification stage (103)), UV radiation treatment stage (105), device (106) for determining total organic content, polisher (107), optional further conductivity cell (108), and is then re-cycled via the recycle line (R), i.e. with valve (110) being open and valve (109b) being closed, to a location upstream of pump (102) and downstream of water inlet (101).

In measuring mode, the total organic carbon content is determined by device (106) to determine the total organic carbon content of the purified water.

Subsequent to the determination of the total organic carbon content, the water purification system (100) of FIG. 1 is preferably switched (manually or automatically) into re-cycling mode, depicted in FIG. 5c, wherein in the water purification system (100) the water follows essentially the same flow as in the dispending mode shown in FIG. 5a but instead of being dispensed via dispensing section (109), the now purified water is re-cycled via the recycle line (R) to the beginning of the water purification flow, i.e. downstream of water inlet (101) and upstream of pump (102).

It is noted that for the water purification system of FIG. 2 the water flow in dispensing, measuring, and recycling mode is essentially similar to that of the purification system (100) of FIG. 1, the only difference being that for measuring mode water flow is controlled to by-pass the first purification stage by closing valve (111a) and opening valve (111b), rather than using two- or three-way valve (111).

Similarly, FIGS. 6a, 6b and 6c show a schematic exemplary representation of the water flow in the water purification system (100) of FIG. 3 in dispensing, measuring, and re-cycling mode, respectively. It is noted that in FIGS. 6a, 6b and 6c dispensing section (109), particularly the parts of dispensing section (109) forming part of the recycle flow as described above, are not shown in detail.

FIG. 6a shows a schematic exemplary representation of the water flow of the water purification system (100) of FIG. 3 in dispensing mode. Feed water is fed into water purification system (100) via water inlet (101), from where the water sequentially passes through the purification stage (103), UV radiation treatment stage (105), device (106) for determining the total organic carbon content, polisher (107), an optional conductivity cell (108), and then into dispensing section (109), from where the purified water is dispensed.

Once dispensing is terminated, the water purification system (100) of FIG. 3 is switched (manually or automatically) into measuring mode as shown in FIG. 6b by opening valve (110) and closing dispensing valve (109b), wherein water then flows through dispensing section (109), through the (open) valve (110) and the recycle line (R), through connecting line C2 comprising circulation pump (112) (thereby by-passing the first purification stage (103)), UV radiation treatment stage (105), device (106) for determining total organic carbon content, and through optional conductivity cell (108) to dispensing section (109).

In measuring mode, the total organic carbon content is determined using device (106) to determine the total organic carbon content of the purified water.

Subsequent to the determination of the total organic carbon content, the water purification system (100) of FIG. 3 is preferably switched (manually or automatically) into re-cycling mode, depicted in FIG. 6c, wherein in the water purification system (100) the water follows essentially the same flow as in the dispensing mode shown in FIG. 6a but instead of being dispensed via dispensing section (109), the now purified water is re-cycled via the recycle line (R) to the beginning of the water purification flow, i.e. downstream of water inlet (101) and upstream of pump (102).

### EXAMPLES

The following examples are intended to illustrate in a non-limiting way the workings and advantages of the present water purification system and the process of operating such.

### Example 1

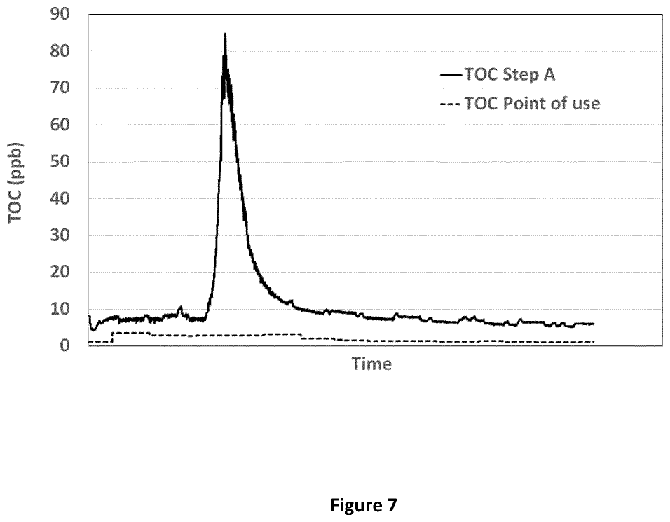

A water purification system as schematically shown in FIG. 1 or FIG. 2 with a Q-Gard® T1 purification cartridge in the first purification stage and a Quantum® TEX polishing cartridge in the second purification stage, both of which are available from Merck KGaA (Darmstadt, Germany), and using feed water from a reverse-osmosis purification stage, was re-started after having sat idle over a weekend. As shown in FIG. 7, shortly after restart a spike in the total organic carbon level was registered by device (106) in the purified water coming out of the first purification stage (see continuous line marked “TOC step A”), while the respective control measurement for dispensed water still showed it to have a total organic carbon content well within specification (see dashed line marked “TOC Point of use”).

These results indicate that relying on the total organic carbon content determined following the first purification stage only may lead to false alarms, i.e. might indicate the dispensed water to not respond to the purity requirements even though in reality it does.

### Example 2

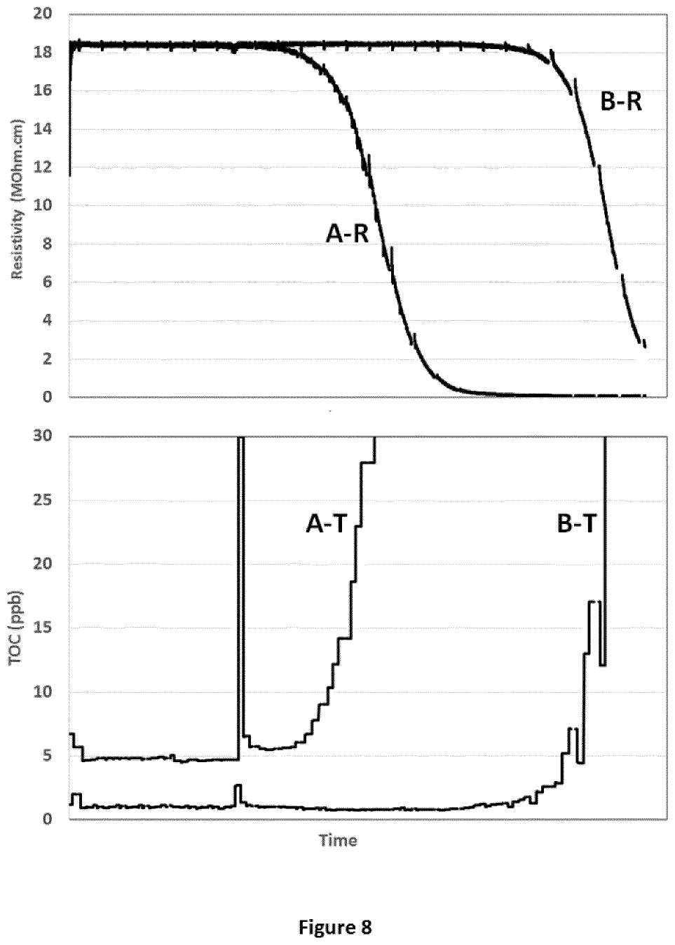

The water purification system of Example 1, with newly installed purification cartridges, was run alternatingly in dispensing mode and in measuring mode until the first and the second purification stages were both exhausted. The respective curves for the resistivity measured by device (106) for determining the total organic content for the water purification flow at device (106), i.e. after having passed the first purification stage, denoted “A-R”, and for the purified water at the point of use, i.e. in the dispensing section, denoted “B-R”, are shown in FIG. 8 together with the respective values for total organic carbon content for the purification flow, denoted “A-T” and for the purified water at the point of use, denoted “B-T”.

The graphs in FIG. 8 clearly show that the total organic carbon content for the water purification flow at device (106) generally indicates a value that is higher than the actual one at the point of use, and further that the water purification system in its entirety, i.e. first and second purification stages together, as shown by curves B-R and B-T, was still able to supply purified water of the required purity even after performance of the first purification stage started to drop as shown by curves A-R and A-T. It can even be seen that for some period when the first purification stage had been completely exhausted the second purification stage alone managed to produce purified water of the desired purity.

Because the present water purification system allows separately determining performance of the first purification stage, the user is alerted in good time to prepare replacement of the first purification stage, and in due time also of the second purification stage. This effectively allows the user to further extend the useful lifetime of the purification cartridges used in the purification stages without running the risk of not being able to produce purified water of the desired purity.

### Example 3

For the water purification system of Example 1 total organic carbon content was determined using device (106) in dispensing mode to yield the “intermediate TOC content” and in measuring mode to yield the “TOC at point of use”. Comparative values were obtained using two reference calibrated TOC monitors, with the first of these withdrawing water directly downstream of the first purification stage, thus reflecting the “intermediate TOC content” and the second of these withdrawing water downstream of the polisher, thus reflecting the “TOC at point of use”. The results of this comparison under various conditions are given in the following Table 1, wherein for the values for “TOC at point of use” and “Comparative TOC at point of use” were obtained for
examples 3.1 and 3.2 directly after having changed the purification cartridges and having flushed them with 20 l of water,
examples 3.3, 3.4, and 3.5 in normal operation after having run the present water purification system in measuring mode for 3 minutes, and
examples 3.6 and 3.7 in normal operation after having run the present water purification system in measuring mode for 10 minutes.

TABLE 1

| | | | Comparative | Comparative |
|---|---|---|---|---|
| | Intermediate | TOC at point | intermediate | TOC at point |
| Example | TOC | of use | TOC | of use |
| 3.1 | 14.3 | 7.9 | 12.3 | 8.7 |
| 3.2 | 13.8 | 5.5 | 11.8 | 6.4 |
| 3.3 | 11.2 | 4.5 | 10.9 | 4.2 |
| 3.4 | 11.8 | 4.9 | 11.6 | 3.6 |
| 3.5 | 9.2 | 4.2 | 8.2 | 3.4 |
| 3.6 | 10.7 | 4.1 | 9.8 | 2.7 |
| 3.7 | 11.2 | 4.7 | 12.4 | 2.8 |

The above data clearly shows that the present water purification system yields reliable values for the total organic carbon content in the purification flow and particularly at the point of use.

### Example 4

Example 2 was repeated, however, with an IPAK Meta® polishing module in the first purification stage and an IPAK Quanta® polishing cartridge in the second purification stage, both of which are available from Merck KGaA (Darmstadt, Germany).

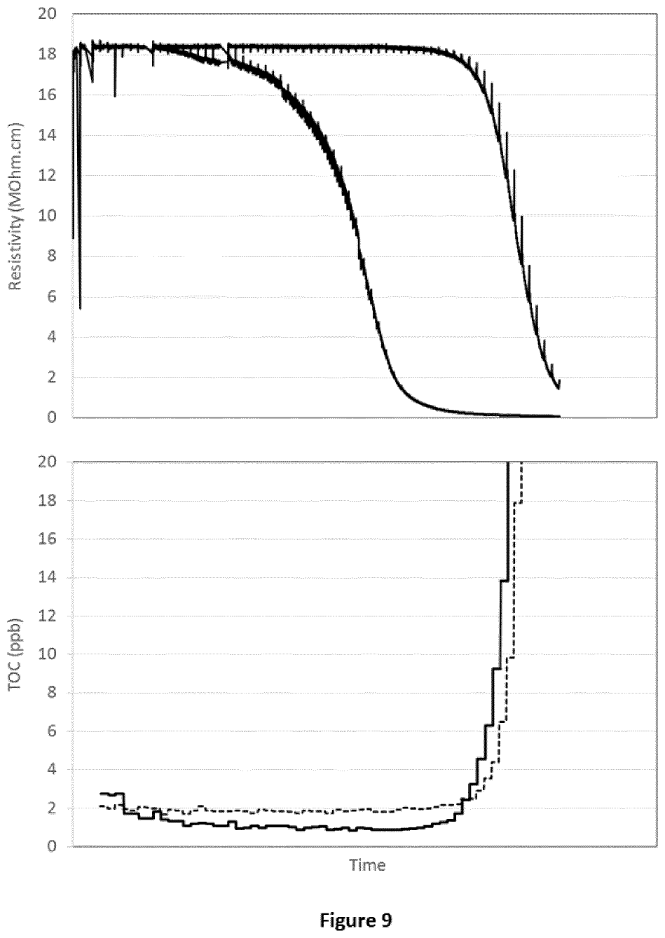

The respective results are shown in FIG. 9 with “A-R” indicating resistivity measured by device (106) and “A-T” denoting the associated total organic carbon content, “B-R” indicating resistivity measured at the point of use and “B-T” denoting the associated total organic carbon content. The curves of FIG. 9 confirm the findings of Example 2.

Overall, the present inventors have very surprisingly found that generally the present water purification system and the present process for operating such give values of total organic carbon content that much better than the previously described water purification systems and methods reflect the total organic carbon content at the point of use, i.e. of the dispensed water.

Furthermore, the present water purification system and process for operating such profits from a specific limitation of such water purification systems as defined herein, namely that the first and second purification stage are generally “symmetrical”, i.e. are of essentially the same size and generally have comparable, i.e. essentially the same, throughput. Hence, the present water purification system and process of operating such allows to specifically distinguish between exhaustion of the first purification stage, i.e. when the first purification stage no longer purges contaminants to the desired extent, and exhaustion of the second purification stage, i.e. when the second purification stage no longer purges contaminants to the desired extent. In case of the first purification stage being exhausted, the second purification stage may very well still be capable of purifying water to the desired purity, thereby allowing purified water of the desired purity to be dispensed. Thus, the present water purification system and process for operating such also allow to better and more precisely determine when a purification stage will need to be replaced. This will allow for reduced operating costs as well as less waste from unnecessarily replaced purification stage hardware, such as purification cartridges.

Thus, the present water purification system and process for operating such allows to overcome at least two drawbacks of the prior art.

The present water purification system also shows surprising versatility and capability in evaluating and determining the individual efficacies of the first purification stage and the second purification stage.

For example, the efficacy of the first purification stage can be determined by first running the present water purification system in dispensing mode (see, for example, FIGS. 5a and 6a) and then switching into measuring mode (see, for example, FIGS. 5b and 6b). Determining conductivity and thereby total organic carbon content first in dispensing mode and then in measuring mode will show the user whether the first purification stage still works correctly and is not exhausted. Namely, if the values in dispensing mode and measuring mode do not differ significantly, the first purification stage still functions as it should. On the other hand, if the values in dispensing mode and measuring mode differ significantly, particularly if the value in dispensing mode is significantly higher than that in measuring mode, the first purification stage does not work as it should and consequently will need replacing.

Additionally, the present water purification comprising a connecting line (represented, for example, by line C1 in FIGS. 1 and 2) liquidly connecting the water purification flow from a branching point, located downstream of the pump and upstream of the first purification stage, to a location downstream of the first purification stage and upstream of the second purification stage allows determining the purity of the feed water arriving via the water inlet into the water purification flow. This may be done by first passing water from the water inlet through the pump and line C1 directly to the second purification stage, where a first conductivity and consequently first total organic content is determined. The water purification system is then switched into measuring mode and a second conductivity and consequently second total organic content is determined. Comparing first and second value will give an indication of the quality of the feed water arriving through the water inlet.

In summary, the present water purification system allows to determine the total organic carbon content of the dispensed purified water with good accuracy and reliability, while at the same time allowing for a simplified and easier to maintain and to manufacture overall water purification system by taking advantage of features that are already present in the water purification stream and putting them to double use.

1. Water purification system comprising:
(a) a purification stage comprising, in sequence a UV radiation treatment step and a polisher; and
(b) a device for measuring total organic carbon content, the device located downstream of the UV radiation treatment step and upstream of the polisher,
(c) a measurement loop,
wherein the measurement loop comprises the purification stage and the device for measuring total organic carbon.

2. Water purification system according to claim 1 comprising in sequence:
(a′) a water inlet;
(b′) a water purification flow comprising in sequence:
(b1′) a pump;
(b2′) a first purification stage for purifying water to a first water purity grade;
(b3′) a second purification stage comprising, in sequence the UV radiation treatment step and the polisher for purifying water to a second water purity grade higher than the first water purity grade; and
(b4′) a dispensing section; and
(c′) a recycle line (R) liquidly connecting the water purification flow from a location downstream of the dispensing section to a location downstream of the water inlet and upstream of the pump;
wherein the water purification flow and/or the recycle line (R) is/are adapted to allow by-passing the first purification stage, thereby establishing the measurement loop.

3. Water purification system according to claim 2, wherein the water purification system comprises a connecting line C1 liquidly connecting the water purification flow from a branching point, located downstream of the pump and upstream of the first purification stage, to a location downstream of the first purification stage and upstream of the second purification stage, through which connecting line the flow of water can by-pass the first purification stage.

4. Water purification system according to claim 3,
wherein the water purification system comprises at the branching point a two-way valve or three-way valve; or
wherein the water purification system comprises a first valve being located in the water purification flow downstream of the branching point and upstream of the first purification stage, and a second valve being located in connecting line C1.

5. Water purification system according to claim 2, wherein the water purification system comprises a connecting line C2 liquidly connecting the recycle line (R) to the water purification flow at a location downstream of the first purification stage and upstream of the second purification stage, and wherein connecting line C2 preferably comprises a circulation pump.

6. Water purification system according to claim 1, wherein the purification stage comprises one or more purification media selected from the group consisting of cationic ion exchanger, anionic ion exchanger, and activated carbon.

7. Water purification system according to claim 1, wherein the UV radiation treatment step comprises a UV radiation source emitting radiation, such UV radiation source being selected from the group consisting of low-pressure mercury lamp, cold cathode mercury lamp, and excimer lamp.

8. Water purification system according to claim 1, wherein the polisher comprises one or more selected from the group consisting of activated carbon, cationic ion exchanger, and anionic ion exchanger.

9. Process for operating a water purification system, the water purification system comprising:
(a) a purification stage comprising, in sequence a UV radiation treatment step and a polisher; and
(b) a device for determining total organic carbon content, the device located downstream of the UV radiation treatment step and upstream of the polisher,
(c) a measurement loop,
wherein the measurement loop comprises the water purification stage and the device for measuring total organic carbon,
wherein the process comprises the steps of
(1) passing water through the purification stage, thereby obtaining purified water; and
(2) passing the purified water through the measurement loop and determining by means of the device the content of total organic carbon comprised in the purified water.

10. Process for operating a water purification system according to claim 9, the water purification system comprising:
(a′) a water inlet;
(b′) a water purification flow comprising in sequence:
(b1′) a pump;
(b2′) a first purification stage for purifying water to a first water purity grade;
(b3′) a second purification stage comprising, in sequence the UV radiation treatment step and the polisher for purifying water to a second water purity grade higher than the first water purity grade; and
(b4′) a dispensing section; and
(c′) a recycle line (R) liquidly connecting the water purification flow from a location downstream of the dispensing section to a location downstream of the water inlet and upstream of the pump;
wherein the process comprises the steps of
(1′) providing water via the water inlet;
(2′) passing water through the water purification flow, thereby obtaining purified water;
(3′) dispensing the purified water obtained in step; and
(4′) determining the level of total organic compound by passing purified water through the measurement loop by by-passing the first purification stage and determining the content in total organic carbon by means of device for measuring total organic carbon.

11. Process for operating a water purification system according to claim 10, the water purification system comprising a connecting line C1 liquidly connecting the water purification flow from a branching point, located downstream of the pump and upstream of the first purification stage, to a location downstream of the first purification stage and upstream of the second purification stage, through which connecting line C1 the flow of water can by-pass the first purification stage,
wherein in step (4′) the first purification stage is by-passed by blocking the water purification flow downstream of the branching point and upstream of the first purification stage, and allowing water to pass through connecting line C1 to the second purification stage.

12. Process for operating a water purification system according to claim 11, the water purification system comprising at the branching point
(i) a two-way valve or three-way valve, or
(ii) a first valve being located in the water purification flow downstream of the branching point and upstream of the first purification stage, and a second valve being located in connecting line C1.

13. Process for operating a water purification system according to claim 10, the water purification system comprising a connecting line C2 liquidly connecting the recycle line (R) to the water purification flow at a location downstream of the first purification stage and upstream of the second purification stage, and the connecting line C2 preferably comprising a circulation pump, wherein in step (4′) the first purification stage is by-passed by forcing water to pass through connecting line C2.

14. Process for operating a water purification system according to claim 9, wherein the purification stage comprises one or more purification media selected from the group consisting of cationic ion exchanger, anionic ion exchanger, and activated carbon.

15. Process for operating a water purification system according to claim 9, wherein the process subsequently to step (4′) comprises a step of re-cycling the purified water by passing it sequentially through the water purification flow and the recycle line (R).

16. Process for operating a water purification system according to claim 9, further comprising a UV radiation source emitting radiation.

17. Process for operating a water purification system according to claim 9, wherein the polisher comprises one or more of activated carbon, cationic ion exchanger, and anionic ion exchanger.
